(19) United States
(12) Patent Application Publication
(10) Pub. No.: US 2011/0300220 A1
(43) Pub. Date: Dec. 8, 2011

(54) SOLID CALCIUM LACTATE IN SUBSTANTIALLY SPHERICAL FORM

(75) Inventors: Philippe Coszach, Courcelles (BE); Jean-Christophe Bogaert, Legne (BE); Emmanuelle Martin, Villeneuve d'Ascq (FR)

(73) Assignee: GALACTIC SA, Escanaffles (BE)

(21) Appl. No.: 13/141,917

(22) PCT Filed: Nov. 18, 2009
(86) PCT No.: PCT/EP09/65397

§ 371 Date: Aug. 24, 2011

(30) Foreign Application Priority Data
Dec. 24, 2008 (BE) 2008/0701

(51) Int. Cl. A61K 9/14; A61P 3/02; A61K 31/191

(52) U.S. Cl. 424/489; 514/557

(57) ABSTRACT

Solid calcium lactate in the form of substantially spherical particles, characterised in that the spherical particles have a particle size distribution such that most of the particles are between 280 and 550 microns in size and the calcium lactate can be rapidly dissolved in water.

### CROSS-REFERENCE TO RELATED APPLICATION

This application is a National Stage filing, under 35 U.S.C. §371, of International Application Serial No. PCT/EP2009/065397, filed Nov. 18, 2009 and designating the United States, which claims priority to Belgium Application Serial No. 2008/0701, filed Dec. 24, 2008, the entire disclosures of which are expressly incorporated herein by reference.

### FIELD OF THE INVENTION

The present invention relates to a solid calcium lactate composition, in the form of substantially spherical particles, having a particle size such that the majority of the particles are between 280 and 550 microns in size; the present invention also relates to a method for obtaining this type of calcium lactate.

### BACKGROUND OF THE INVENTION

Calcium lactate or calcium hydroxy-2-propanoate is the calcium salt of lactic acid, a natural acid produced by bacterial fermentation on starch or molasses. It exists in various forms: anhydrous, monohydrate, trihydrate and pentahydrate. Calcium lactate has particularly suitable physico-chemical characteristics for enriching a wide range of food products and for calcium supplementation.

However, whether for supplementation or for enriching food products, there is a genuine need for a source of calcium with enhanced properties, particularly superior resistance to attrition to prevent the formation of fines, enhanced flow properties and additionally to be able to dissolve said calcium lactate rapidly and completely for easy use and consumption.

Calcium, found in mineral salt form in the body, represents 2% of body weight. 99% is found in the bones and teeth where it provides rigidity and strength. The remaining percentage (1%) circulates in the blood and plays a role in the proper functioning of the nervous and cardiovascular systems. If an insufficient amount of calcium is circulating in the blood, it is replenished by calcium from the bones. The bones thus continually lose calcium.

To prevent deficiencies, sources of growth retardation, bone fragility and osteoporosis, calcium needs to be “restocked” via the diet.

Milk and dairy products (infant formulas, cheeses and yogurts) are currently the main source of calcium in the diet for many populations. However, many subjects encounter difficulties meeting their daily requirements via this type of products. A number of reasons explain this phenomenon, for the following reasons:
cultural
sociological: choice of low-fat diets, vegan diets
physiological: disorders associated with dairy products, such as digestive problems and allergies

A number of possibilities enabling subjects to increase their calcium intake are now well-known. They are based on a change of diet or on the consumption of nutritional supplements or calcium-enriched products.

The flowability and percentage of fines in the ingredients used in tablet formulation are important parameters for the production of this dosage form. Indeed, powders having a high degree of flowability offer the advantage of being able to sustain high production speeds. Similarly, reducing the electrostatic and pneumatic coating of fines helps increase yields and keeps chambers and machines clean.

However, sources of calcium are most frequently in the form of a powder having a relatively non-uniform particle size distribution and containing a high percentage of fines. These features limit the flowability of the powder and thus the use thereof in tablet production.

Supplementation in calcium powder form may also be offered to consumers. These powders generally contain calcium carbonate or calcium citrate, which are slightly soluble in water. The consumer also generally needs to wait patiently for the powder to dissolve before being able to consume the product. Otherwise, the non-dissolved particles will be liable to reduce the organoleptic qualities of the solution and the uptake thereof by the body considerably.

For these various reasons, consumers are turning to enriched products. Enrichment, which consists of incorporating calcium in the composition of some foods is thus perceived by consumers to be an easy way to increase their consumption. To meet these expectations, the food processing industries can use a wide range of sources of calcium. The selection of the suitable source is generally based on various criteria associated with the finished product, such as solubility, calcium content, taste and bioavailability. Other criteria relating to the ease of use and the cost of the ingredient are also taken into account. Indeed, enriching a food product with minerals frequently increases the overall production cost for industrial manufacturers. Calcium salts such as calcium citrate or calcium malate requiring a long dissolution time or a high dissolution temperature generally incur additional costs. Moreover, raw materials containing high quantities of dust incur significant losses for the business (the quantity of calcium actually delivered to the consumer may be lower than expected for this reason).

However, at the present time, known compositions include, in addition to calcium lactate, a non-negligible quantity of calcium citrate or malate, but this nonetheless does not produce the desired properties, in view of the deficiency thereof in terms of solubility in water, but also due to the impact thereof on the taste of the product.

Moreover, the majority of sources of calcium, including calcium lactate, are found in the form of particle clusters highly susceptible to the attrition phenomenon and thus liable to limit the use thereof.

The various forms of calcium lactate available on the market are generally obtained by mass crystallisation followed by grinding or spray drying and, in any case, the particle size is not suitable, and the shapes of the granules are far from substantially spherical, which is not desired to meet the aims of the invention.

In the case of mass crystallisation followed by grinding, the ingredient obtained consists of particles having poor handling properties (powdery fines) and flow properties (irregularly shaped particles in particular).

Spray drying which consists of spraying a liquid in a mist of fine droplets and placing said fine droplets in contact with hot air in a drying chamber generates an ingredient having a particle size distribution rich in powdery, difficult to dissolve fines.

Despite the good general solubility of calcium lactate, the forms obtained by grinding or by spray drying are difficult to “solubilise” at ambient temperature due to the large quantity of fines contained therein.

Attempts have already been made to remedy these drawbacks particularly in the description of the patent US2008152764 but with no significant success. Methods for obtaining solid calcium lactate, as in the patent WO 0028973 have already been described, but they relate to powders in which the sizes do not exceed 40 microns, to agglomerate same with a binder to obtain tablets.

There is thus a need for preparing and obtaining a solid calcium lactate, in the form of particles having a spherical shape offering the advantage of a much higher resistance to the attrition phenomenon which limits the use of calcium particle clusters, thereby generating a lower percentage of fines and increasing the dissolution rate of the ingredient. Additionally, there is a need for solid calcium lactate, in the form of particles, having a particle size distribution such that the presence of particles less than 150 microns in size is limited very significantly and that said calcium lactate can be dissolved rapidly and readily and such is the aim of the present invention.

The invention also relates to a method for obtaining solid calcium lactate particles, in the form of substantially spherical particles.

### BRIEF SUMMARY OF THE INVENTION

The present disclosure relates generally to a solid calcium lactate in the form of substantially spherical shaped particles. According to an embodiment of the present disclosure, the solid calcium lactate has a higher resistance to the attrition phenomenon than calcium particle clusters, and thereby generates a lower percentage of fines. According to another embodiment, the solid calcium lactate of the present disclosure also includes an increased dissolution rate and has a particle size distribution such that the presence of particles less than 150 microns in size is significantly limited, such that said calcium lactate can be dissolved rapidly and readily.

According to an exemplary embodiment of the present disclosure a solid calcium lactate in the form of spherical particles having a dissolution rate, measured using the METHOD, of less than or equal to three minutes is disclosed. The sodium lactate has a particle size distribution such that greater than fifty percent of the particles are between 280 and 550 microns in size.

According to yet another exemplary embodiment of the present disclosure, the solid calcium lactate particles may have a particle size distribution such that the percentage of particles less than 280 microns in size does not exceed 40%.

In another embodiment of the present disclosure, a method for obtaining a solid calcium lactate in the form of substantially spherical particles is provided. The solid calcium lactate has a particle size distribution such that the majority of the spherical particles are between 280 and 550 microns in size. The method includes, in a first step, a calcium lactate solution being atomized in a fluidised bed granulator at an incoming air temperature of less than 80 degrees Celsius and the wet spherical calcium lactate particles being retrieved. The method also includes, in a second step, the wet spherical particles undergoing a heat treatment in a fluidised bed at an incoming air temperature of less than 165 degrees Celsius.

According to another exemplary embodiment of the instant disclosure, during the first step the calcium lactate introduced into the granulator has a concentration between 20 and 60%.

According yet to another embodiment of the instant disclosure, the degree of moisture of the calcium lactate particles obtained in the first step does not exceed 45%.

### DETAILED DESCRIPTION OF THE INVENTION

The Applicant has now unexpectedly discovered that, by producing solid calcium lactate, in the form of substantially spherical particles having a particle size distribution such that the presence of particles less than 150 microns in size is limited very significantly and capable of dissolving rapidly in water, it was possible to remedy the drawbacks of the compositions according to the prior art.

In one of the embodiments of the invention, the calcium lactate solution having a concentration capable of varying between 20 and 60% by weight is fed by spraying in a fluidised bed where calcium lactate fines are kept in suspension. The air flow rate (to keep the bed suspended at the spray nozzles), calcium lactate flow rate and concentration and temperature conditions are controlled so as to enable particle size growth essentially by layer (onion structure) and not by the agglomeration of these fines.

In general, according to the method according to the invention, calcium lactate concentrations between 20% and 50%, an air temperature and the spray chamber inlet less than 80° C. are used to obtain particles in which the sizes grow by layer but which eventually have a particle size distribution such that 50 to 80% of the particles are between 280 and 550 microns in size.

One preferential process for using the technology consists of producing spherical lactate beads in two steps. The first (granulation-crystallisation) performed at a low temperature will consist of forming porous beads by spraying in a fluidised bed consisting of fines and the second of bringing same to the desired moisture level. The second step may be performed in a fluidised bed or by using any other technology known to a person skilled in the art. By adapting the temperature and residence time of the product in the drying chamber (from a few minutes to several hours), it is possible to obtain a calcium lactate in pentahydrate (between 22 and 27% moisture) or trihydrate (between 15 and 20% moisture) or monohydrate (between 5 and 8% moisture) or anhydrous (less than 3% moisture) form.

Unlike the method described in the invention, the spraying technologies conventionally used make use of high incoming air temperature intended to achieve a low product moisture content rapidly. In this case, the finished products generally include a high proportion of fines. One alternative embodiment of the conventional method makes it possible to obtain agglomerated products with larger particle sizes. After partial spray drying at incoming air temperatures above 120° C., the particles are agglomerated in a porous layer on a conveyor drier. A final crushing or crumbling step is then required to obtain a granulated powder having the desired sizes.

One preferential process for obtaining spherical granulated particles directly (not requiring agglomeration followed by crushing) is the use of fluidised bed granulation technology. However, this method requires that the particles “floating” in the air of the fluidised bed do not tend to stick together. The use of high temperatures favours the particle adhesion phenomenon and gives rise to significant lumpiness liable to block the facility.

Unexpectedly, the applicant has now discovered that, by working at a low temperature, calcium lactate can be kept in a non-adhesive crystalline form and thus ensure satisfactory particle fluidisation.

The Applicant has now discovered that, by applying these conditions, the product obtained has an enhanced resistance to attrition, flow properties and dissolution rate and a high bulk density (“aerated” as opposed to “packed”).

Flowability.

Flowability can be defined as the ability of a powder to flow regularly under the effect of gravity or any other force. Various powder flow measurement methods have been documented in the literature. The comparative analysis of various analysis methods conducted by J. L. Ilari (Ilari J. L., Flow properties of industrial dairy powders, Tech. Lait 82 (2002) 383-399.) led to the recommendation of the Hosokawa physical property analyser method. This method, which is particularly suitable for analysing the flow of dairy powders, may also be applied to calcium lactate powders.

The Hosokawa analyser thus makes it possible to measure seven main physical characteristics on powders, relating to the flowability thereof and also the slidability. The seven characteristics measured are described in the manual published by Hosokawa Micron Corp (Hosokawa Micrometrics Laboratory, Manual for the use of Hosokawa powder tester, Hosokawa Micron Corp., Osaka, Japan, 1981. A brief description of the measurements made is given below:
the angle of repose consisting of measuring the angle of a pile of particles previously dispersed by a vibrating screen and collected by a funnel and placed on a work surface.
the drop angle consisting of measuring the angle of said pile of powder after applying a shock thereto (vertical drop from a fixed height of an object of predetermined mass)
on the basis of the drop angle and the angle of repose, the angle of difference is deduced,
the apparent density (A): the measurement is obtained by weighing a quantity of powder collected in a container of known volume
the packed density (P): the same weighing is performed after packing the powder (180 vertical drops of the container from a predefined height); from these two density measurements, the compressibility C (C=100*(P−A)/P) of the powders can be deduced. The compressibility is a particularly important factor for calculating the flow index. If the compressibility value is greater than 20%, the powder is considered to be “non-flowing”.
the angle of spatula: the angle of a pile of powder placed on a spatula is measured,
uniformity: the powder passes through three stacked vibrating screens and the method consists of weighing the quantity of powder “retained” by each screen after a predetermined vibration time. This parameter is a numeric value obtained by dividing the width of the pores of the screens allowing 60% of the powder to pass by the width of the pores of the screens allowing 10% of the powder to pass. The greater the uniformity of the powder, the closer the ratio described above is to 1.
dispersion: a watch-glass is positioned perpendicular to a hatch whereon the powder is unloaded. The quantity of powder collected on the watch-glass after opening the hatch suddenly is determined. This feature also makes it possible to detect the presence of fines in the powder. The greater the quantity of powder collected on the watch-glass, the smaller the quantity of dust and the dispersibility value.

An index is assigned to each physical characteristic measured. The sum of these indexes is used to determine the flow or slide index, corresponding to a degree of flow or slide.

It has now been discovered that, by applying the conditions according to the invention, a calcium lactate having a high flow index greater than 80, or greater than 90, corresponding to a degree of flow deemed to be “quite good”, or even “very good”, was obtained. More specifically, the uniformity and the angle of spatula of the calcium lactate described in the invention have values less than 2 and 30°, respectively. A uniformity value greater than 2 and an angle of spatula value greater than 30° lower the flow index and thus reduce the ease of use of calcium lactate. At the present time, these types of product are encountered on the market.

Finally, the calcium lactate described in the invention has a high flow index greater than 60, corresponding to a degree of slide deemed to be “quite good” and superior to that currently found on the market.

Particle Size Distribution.

When moving, all powders are subject to the attrition phenomenon giving rise to the formation of undesirable residue. In various experiments, it was highlighted that particles having a particle size distribution less than 100 microns had a slower dissolution rate in water than that of particles having a size between 150 and 710 microns in size. In order to increase the dissolution rate of calcium lactate, it was thus important to remove these particles and the formation thereof.

In known methods, a final porous agglomerate grinding, crushing or crumbling step is used, giving rise to variable forms liable to affect the flow properties of the product and generating a number of fines. The applicant has now discovered that, by removing the grinding, crushing or crumbling step, and by applying the conditions of the invention, more spheroid forms are obtained, with which the quantity of fines is limited and a more advantageous particle size distribution is obtained.

The attrition phenomenon may be demonstrated as follows: place 100 g±0.01 g of the test sample (for which the particle size distribution obtained using the screening method is known) on a screen wherein the mesh size of the metal cloth is 100 microns and activate the Retsch AS200 vibrating screen with an amplitude 0.95 mm/´ǵ. The screening time is set to 1 hour.

After switching off the screen, collect the 100 g of sample subject to the attrition phenomenon and analyse the particle size distribution thereof using a conventional measurement apparatus. Compare the particle size distribution after attrition to that obtained before attrition.

The particle size distribution of the samples susceptible to attrition varies as follows: the quantity of small particles increases whereas that of large particles decreases.

Note: particles less than 100 microns in size are previously removed from the sample to undergo the attrition test. The particle size distribution of the sample before the attrition test accounts for this change.

Dissolution Rate.

For the purposes of the present patent application, the dissolution rate is determined by the following method, hereinafter defined by the term “METHOD”: in a 100 ml volume of deionised water kept at 25° C.±1° C. in a 500 ml beaker equipped with a bar magnet rotating at a controlled speed of 400 rpm±20 rpm, 5 g±0.01 g of calcium lactate sample is introduced. A timer is started at the precise moment when the product is added and stopped as soon as the last particle disappears/is solubilised. The dissolution rate is then represented by the time required for the dissolution of the entire sample.

The calcium lactate described in the invention offers the specificity of being both sufficiently robust to offer a high attrition resistance and porous to enable rapid dissolution (dissolution rate less than 3 minutes).

### EXAMPLES

The example hereinafter are given to illustrate the invention, without limiting the scope thereof.

### Example 1

A calcium lactate pentahydrate powder was prepared in two separate steps.

During the first step, a 32% calcium lactate solution was atomised in a fluidised bed granulator continually supplied with fines. The bottom screen of the fluidised bed has a surface area of 0.45 m². The air temperature at the atomisation chamber inlet may fluctuate but remains between 52 and 55° C. The incoming air flow rate is adjusted to 1700 m³/hr.

At the end of this first step of the method, a wet product in the form of substantially spherical particles is obtained.

In a second step of the method, the wet particles obtained in the first step then undergo a heat treatment to bring the calcium lactate to the desired degree of moisture. This second step is performed in a fluidised bed for which the incoming air temperature is 125° C.

Following the second step of the method, a product, still in the form of substantially spherical particles and having a moisture content of approximately 23%, is obtained, for which the physicochemical properties are given in Table 1 and the particle size distribution is given in Table 2.

As a comparative example, a calcium lactate powder was prepared using a routine method and a powder was obtained for which the physicochemical properties are specified in Table 1 and the particle size distribution is given in Table 2.

TABLE 1

| | Calcium lactate | Calcium lactate |
|---|---|---|
| | described in the | obtained according to |
| Characteristic | invention | the prior art |
| Calcium concentration | 13.7 g of calcium/ | 13.7 g of calcium/ |
| | 100 g of calcium | 100 g of calcium |
| | lactate | lactate |
| Angle of spatula | 27.7° | 30.6° |
| (Hosokawa Powder | | |
| Characteristics Tester) | | |
| Angle of repose (Hosokawa | 33.6° | 32.6° |
| Powder Characteristics | | |
| Tester) | | |
| Uniformity (Hosokawa | 1.55 | 1.98 |
| Powder Characteristics | | |
| Tester) | | |
| Drop angle (Hosokawa | 23.6° | 25.8° |
| Powder Characteristics | | |
| Tester) | | |
| Angle of difference | 10.0° | 6.8° |
| (Hosokawa Powder | | |
| Characteristics Tester) | | |
| Dispersibility (Hosokawa | 13.8 | 14.0 |
| Powder Characteristics | | |
| Tester) | | |
| Compressibility (Hosokawa | 2.4% | 6.6% |
| Powder Characteristics | | |
| Tester) | | |
| Flow index (Hosokawa | 94.0 | 89.5 |
| Powder Characteristics | (a value greater than | |
| Tester) | 90 indicates a very | |
| | good flow) | |
| Slide index (Hosokawa | 67.00 | 62.75 |
| Powder Characteristics | | |
| Tester) | | |
| Dissolution rate | 2 min 50 sec | 3 min 25 sec |

TABLE 2

| | | Calcium lactate | |
|---|---|---|---|
| | | described in the | Calcium lactate obtained |
| Characteristic | | invention | according to the prior art |
| >710 | microns | 0% | 0% |
| 550-710 | microns | 12% | 0% |
| 400-550 | microns | 42% | 8% |
| 280-400 | microns | 33% | 30% |
| 100-280 | microns | 13% | 59% |
| 0-100 | microns | 0% | 3% |

The calcium lactate described in the invention has a particle size distribution such that 50 to 80% of the particles are between 280 and 550 microns in size. Comparatively, the calcium lactate obtained according to the prior art has a particle size distribution such that more than 55% of the particles are between 0 and 280 microns in size

### Example 2

Two calcium lactate samples were produced in a test cell using a 40% calcium lactate solution using the method described in example 1 below. The only parameter to vary was the incoming air temperature during the first step, which are 52 and 80° C., respectively.

The physicochemical analyses of both samples are given in Table 3 below:

TABLE 3

| | Calcium lactate | Calcium lactate obtained |
|---|---|---|
| | described in the | using the same method |
| | invention | performed at higher |
| | (incoming air | incoming air temperatures |
| Characteristic | temperature 52° C.) | (80° C.) |
| Calcium | 13.7 g of calcium/100 g | 13.7 g of calcium/100 g of |
| concentration | of calcium lactate | calcium lactate |
| Dissolution rate | <3 min | >7 min |

### Example 3

Calcium lactate samples were prepared so as to determine the dissolution rate. For this purpose, 5 g of each of the particle size distribution ranges studied were taken, it being understood that, for the particle size interval ranging from 0 to 100 microns, a sample obtained using the routine method was taken. The dissolution rates as a function of the particle size distribution are given in Table 4 below:

TABLE 4

| Particle size | Dissolution rate |
|---|---|
| 500-710 microns | 2.98 min |
| 425-500 microns | 2.79 min |
| 300-425 microns | 2.38 min |
| 212-300 microns | 2.04 min |
| 150-212 microns | 2.6 min |
| 100-150 microns | 3.08 min |
| 0-100 microns | >7 min |

A powder containing few particles less than 100 microns such as the product described in the invention would thus tend to have a lower dissolution rate. Furthermore, it was observed in table 1 that the dissolution rate of the complete powder obtained according to the prior art was greater than the dissolution rate of the complete powder described in the invention (3 min 25 sec as opposed to 2 min 50 sec).

### Example 4

The particle size distributions of the calcium lactate samples subject to an attrition phenomenon according to the principle described above are shown in Table 5 below.

TABLE 5

| | | Calcium lactate | | Calcium lactate | | Calcium | |
|---|---|---|---|---|---|---|---|
| | | described | | described in the | | lactate obtained | |
| | | in the invention | | invention | | according | |
| | | (product 1) | | (product 2) | | to the prior art | |
| | | Attrition step | | | | | |
| Characteristic | | Before | After | Before | After | Before | After |
| >500 | microns | 37% | 37% | 15% | 15% | 0% | 0% |
| 400-500 | microns | 29% | 29% | 20% | 20% | 2% | 1% |
| 300-400 | microns | 26% | 26% | 35% | 35% | 23% | 19% |
| 100-300 | microns | 8% | 8% | 30% | 30% | 75% | 77% |
| 0-100 | microns | 0% | 0% | 0% | 0% | 0% | 2% |

Unlike calcium lactate obtain according to the prior art, the calcium lactate described in the invention (product 1 and product 2) does not give rise to particles less than 100 microns when subject to an attrition phenomenon. It has a lower susceptibility to attrition than calcium lactate obtained according to the prior art.

1. Solid calcium lactate in the form of substantially spherical particles and having a dissolution rate measured using the METHOD, less than or equal to 3 minutes, characterised in that the solid calcium lactate has a particle size distribution such that greater than 50% of the particles are between 280 and 550 microns in size.

2. Solid calcium lactate, in the form of substantially spherical particles according to claim 1, characterised in that the solid calcium lactate has a particle size distribution such that the percentage of particles less than 280 microns in size does not exceed 40% of the particles.

3. Solid calcium lactate particles according to claim 1, characterised in that the particles have a high resistance to attrition.

4. Solid calcium lactate according to claim 1 characterised in that the spherical particles have a dissolution rate of less than minutes.

5. Method for obtaining solid calcium lactate in the form of substantially spherical particles and having a particle size distribution such that the majority of the spherical particles are between 280 and 550 microns in size, characterised in that:
(i) in a first step, a calcium lactate solution is atomised in a fluidised bed granulator at an incoming air temperature of less than 80° C.;
(ii) the wet substantially spherical calcium lactate particles are retrieved; and
(iii) in a second step, said wet particles undergo a heat treatment in a fluidised bed at an incoming air temperature of less than 165° C.

6. Method according to claim 5 characterised in that, during the first step, the calcium lactate solution introduced into the granulator has a concentration between 20 and 60%.

7. Method according to claim 5 characterised in that the incoming air temperature in the granulator is between 50 and 75° C.

8. Method according to claim 5 characterised in that the incoming air temperature in the second step is between 110 and 160° C.

9. Method according to claim 5 characterised in that the calcium lactate particles obtained in the first step comprise a degree of moisture which does not exceed 45%.
